(19) United States
(12) Patent Application Publication
(10) Pub. No.: US 2022/0330799 A1
(43) Pub. Date: Oct. 20, 2022

(54) SYSTEM AND METHOD FOR USING DETECTABLE RADIATION IN SURGERY

(71) Applicant: Arthrex, Inc., Naples, FL (US)

(72) Inventors: Bruce Laurence Kennedy, Santa Barbara, CA (US); Craig Speier, Santa Barbara, CA (US); Eric Butler, Naples, FL (US); Ryan Kellar, Naples, FL (US); Peter Dreyfuss, Naples, FL (US); John Sodeika, Naples, FL (US); Jake Jolly, Naples, FL (US); Tom Dooney, Naples, FL (US); Reinhold Schmieding, Naples, FL (US)

(73) Assignee: Arthrex, Inc, Naples, FL (US)

(21) Appl. No.: 17/720,443

(22) Filed: Apr. 14, 2022

Related U.S. Application Data
(60) Provisional application No. 63/174,966, filed on Apr. 14, 2021.

(51) Int. Cl. A61B 1/04; A61B 1/045; A61B 1/06; A61B 1/00; A61B 90/00

(52) U.S. Cl. CPC A61B 1/043; A61B 1/045; A61B 1/0638; A61B 1/00045; A61B 90/39; A61B 2090/395

(57) ABSTRACT

A surgical camera system includes a camera with at least one sensor configured to capture image data comprising a first range of wavelengths and a second range of wavelengths. An excitation light source emits an excitation emission at an excitation wavelength. A controller is in communication with the at least one sensor of the camera. The controller is configured to process the image data from the at least one sensor and detect at least one fluorescent portion of the image data in response to a fluorescent emission generated by a fluorescent agent in the second range of wavelengths. The controller is further configured to generate enhanced image data demonstrating the at least one fluorescent portion of the surgical implement in the image data.

### CROSS-REFERENCE TO RELATED APPLICATIONS

This application claims priority under 35 U.S.C. § 119(e) and the benefit of U.S. Provisional Application No. 63/174,966 entitled SYSTEM AND METHOD FOR USING DETECTABLE RADIATION IN SURGERY, filed on Apr. 14, 2021, by Bruce Laurence Kennedy et al., the entire disclosure of which is incorporated herein by reference.

### BACKGROUND OF THE INVENTION

The present disclosure generally relates to a surgical visualization system and, more particularly, to devices and methods utilizing detectable radiation in surgery.

Typically, during endoscopic surgery, a surgical field is cluttered with different anatomical structures and surgical implements as well as fluids that can obscure a surgeon's view of relevant anatomical structures and surgical implements. It is often difficult to see the position of surgical implements relative to different anatomical structures and to properly position surgical instruments in the surgical field. The disclosure provide for various systems and methods to improve the visualization of surgical implemented in surgery settings.

### SUMMARY OF THE INVENTION

In various implementations, the disclosure provides for surgical implements that comprise a fluorescent agent. The fluorescent agents may be incorporated in surgical tools or implements to assist in distinguishing the implements, or portions of the implements, from their surroundings in a surgical field. In general, the fluorescent agents may be excited in response to receiving an excitation emission of radiation over a range of excitation wavelengths. In response to the excitation emission, the fluorescent agent emits a fluorescent emission of radiation in a known wavelength band that is detectable in image data captured by the surgical camera. In response to the detection of the fluorescent emission, the camera may respond in a number of ways to improve the visualization, detection, and/or identification of the surgical implement associated with the fluorescent agent. In some cases, the excitation emission and/or the fluorescent emission may correspond to wavelengths of light capable of penetrating biological tissue. In such cases, the fluorescent emission may be detected by the camera system to identify a position or presence of the surgical implement through the biological tissue. Once identified, a display controller of the camera system may overlay or provide a visual indication of the position of the fluorescent portion of the surgical implement in the image data for improved visualization during surgery. These and other features are described in the following detailed description.

These and other features, objects and advantages of the present disclosure will become apparent upon reading the following description thereof together with reference to the accompanying drawings.

### BRIEF DESCRIPTION OF THE DRAWINGS

The features, aspects and advantages of the present disclosure will become better understood with regard to the following description, appended claims and accompanying figures wherein:

FIG. 1 is a representative diagram of a surgical environment demonstrating a camera system for improved visualization during surgery;

FIG. 2A is a simplified diagram of a camera configured to excite a fluorescent agent and identify a resulting fluorescent emission in a surgical field;

FIG. 2B is a simplified diagram demonstrating a surgical implement illuminated with visible light;

FIG. 2C is a simplified diagram demonstrating the surgical instrument of FIG. 2B enhanced to emphasize a fluorescent portion;

FIG. 3 is a simplified, cutaway diagram demonstrating surgical implements including surgical sutures and anchors comprising a fluorescent agent;

FIG. 4 is a representative diagram demonstrating the sutures and suture anchor of FIG. 3 enhanced by a camera system;

FIG. 5A is a profile view of a shaver comprising a plurality of fluorescent markings configured to identify an orientation;

FIG. 5B is a profile view of a surgical probe demonstrating a plurality of graduated markings identifying a dimension of the surgical probe;

FIG. 6 is a representative diagram demonstrating enhanced image data captured by a surgical camera in a cavity of a patient;

FIG. 7 is a projected view of an arthroscopic operation performed on a shoulder of a patient;

FIG. 8 is a representative diagram demonstrating enhanced image data in a shoulder cavity of a patient;

FIG. 9 is a representative diagram demonstrating enhanced image data in a shoulder cavity of a patient;

FIG. 10A is a projected view demonstrating a surgical procedure for a shoulder;

FIG. 10B is a representative diagram demonstrating a plurality of sutures enhanced with distinctive colors or patterns for improved visualization;

FIG. 11 is a flowchart demonstrating a method of object or surgical implement detection in a surgical field;

FIG. 12 is a flowchart demonstrating a method for providing an enhanced display of surgical image data; and

FIG. 13 is a modified block diagram demonstrating a surgical camera system and display in accordance with the disclosure.

### DETAILED DESCRIPTION

In the following description of the preferred implementations, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

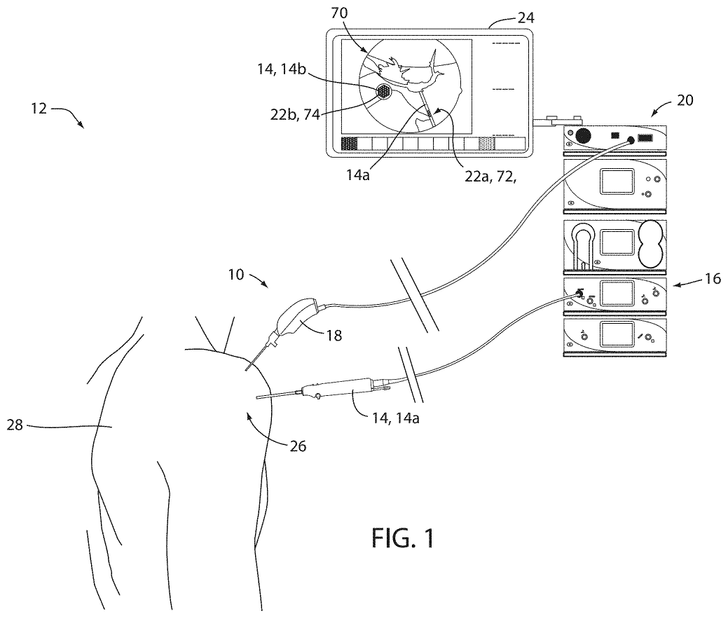

Referring to FIG. 1, a simplified representation of a camera system 10 is shown demonstrating an exemplary surgical environment 12. As shown, the camera system 10 is implemented in combination with one or more surgical implements 14, for example, a surgical tool 14a or shaver in connection with a control console 16. In operation, a camera or endoscope 18 of the camera system 10 may capture image data in a visible light range (e.g., 400 nm to 650 nm) as well as a near-infrared range (e.g., 650 nm to 900 nm). The image data may be communicated to a display controller 20 configured to generate enhanced image data. The enhanced image data may emphasize or visibly define one or more fluorescent portions 22 of the surgical implements 14 to assist in the visualization of one or more of the surgical implements 14 presented on a display device 24. In this configuration, the camera system 10 may provide for improved visualization and enhanced viewing of fluorescent portions 22 of the surgical implements 14 to improve the visibility, detection, and identification of the surgical implements 14 when implemented in a surgical site 26 of a patient 28.

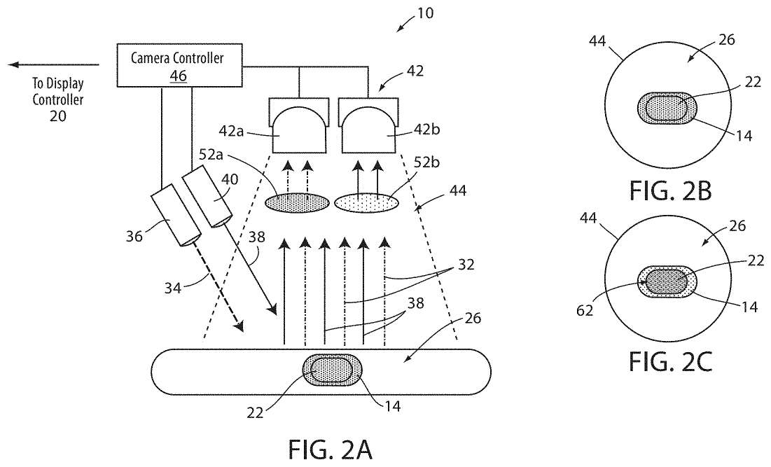

FIGS. 2A-2C are simplified diagrams demonstrating the operation of the camera system 10 to identify a fluorescent emission 32 output from the fluorescent portion of an exemplary surgical implement 14. Referring now to FIGS. 1 and 2A-2C, in various implementations, the fluorescent portions 22 of the surgical implements 14 may comprise a fluorescent agent implemented in a coating, insert, or embedded structure that may become excited and emit the fluorescent emission 32 in response to receiving an excitation emission 34. As demonstrated in FIG. 2A, the excitation emission 34 is output from a first light source 36 and may correspond to an emission of light outside the visible spectrum. Additionally, a visible light emission 38 may be output from a second light source 40. The excitation emission may include a wavelength or range of wavelengths configured to energize and excite the fluorescent agent incorporated in the fluorescent portion 22. In various examples, the excitation emission 34 may comprise wavelengths in a near-infrared range, which may correspond to wavelengths ranging from approximately 600 nm to 900 nm. The first light source 36 may correspond to a laser emitter module configured to output emissions ranging from 650 nm to 680 nm. In some cases, the first light source 36 may output the excitation emission 34 in a range of wavelengths from approximately 740 nm to 780 nm. The specific excitation wavelength associated with the first light source 36 and the excitation emission 34 may be selected to effectively energize the fluorescent agent of the fluorescent portion 22, such that the resulting fluorescent emission 32 may be captured by one or more image sensors 42 of the camera system 10. In this way, the camera system 10 may detect a presence or a location of the surgical implement 14 in response to the detection of the fluorescent emission 32 in the image data.

As previously discussed, the camera system 10 may be configured to capture image data associated with the visible light emission 38 as well as the fluorescent emission 32. Once captured, the system 10 may enhance the image data representing the visible light with one or more overlays or graphics to generate enhanced image data that emphasizes and/or identifies portions of a field of view 44 corresponding to the surgical implement 14. In order to provide the enhanced image data, a camera controller 46 may be configured to selectively control each of the first and second light sources 36, 40 as well as process image data received from a first image sensor 42a and a second image sensor 42b. In a standard operating mode, the camera controller 46 may activate the visible light emission 38 output from the second light source 40 to illuminate the surgical site 26 in wavelengths of light in a visible range (e.g., 400 nm-650 nm). Reflections from the visible light emission 38 may be captured by the second image sensor 42b, which may correspond to a visible light image sensor. Such operation may provide for illumination of the surgical site 26 in visible wavelengths of light, such that the camera controller 46 can output image data demonstrating visible characteristics of the surgical site 26 to the display controller 20. An example of the surgical implement 14 demonstrated illuminated by the visible light emission 38 and captured by the second image sensor 42b is shown in FIG. 2B. Though only a simplified representative body is demonstrated in FIG. 2B to represent the surgical implement 14, the fluorescent portion 22 is represented as being nearly visibly indistinguishable from the depicted surface textures illuminated by the visible light emission 38.

In order to generate the enhanced image data, the camera controller 46 may activate the first light source 36 to output the excitation emission 34. In response to the excitation emission 34, the fluorescent agent of the fluorescent portion 22 may become excited and output the fluorescent emission 32. Concurrent with the activation of the first light source 36, the camera controller 46 may also activate the second light source 40 to illuminate the surgical site 26 in the visible light emission 38. As a result, the fluorescent emission 32 and the visible light emission 38 may be captured within the field of view 44 of each of the image sensors 42. While the second image sensor 42b may be configured to capture the reflected visible light emission 38, the first image sensor 42a may correspond to a near-infrared image sensor configured to capture wavelengths of light in a near-infrared range (e.g., 650 nm-900 nm). As shown, each of the image sensors 42 may comprise one or more light filters, exemplified as a first light filter 52a and a second light filter 52b. In operation, the light filters 52a, 52b may filter the combined wavelengths of the fluorescent emission 32 and the visible light emission 38 in the field of view 44 to improve the fidelity of the detection of the corresponding wavelengths detected by each of the image sensors 42a, 42b. In this way, the camera controller 46 may process image data recorded by each of the image sensors 42a, 42b to detect and discriminate between the fluorescent emission 32 and the visible light emission 38 in the field of view 44 representative of the surgical site 26.

Though generally described as light filters 52, the first filter 52a and the second filter 52b may correspond to one or more high pass, low pass, and/or bandpass filters configured to transmit light over a range associated with a corresponding detection range of the image sensors 42a, 42b. For example, the first light filter 52a may correspond to a bandpass filter configured to pass a range of near-infrared wavelengths from approximately 800 nm to 850 nm. In this configuration, the first light filter 52a may be selected to have a center frequency of approximately 825 nm, which may effectively pass wavelengths of light associated with the fluorescent emission 32 to the first image sensor 42a. In such cases, the fluorescent emission 32 may correspond to an emission from a fluorescent agent in the form of an indocyanine green (ICG) dye. Accordingly, the fluorescent emission 32 output from the fluorescent portion 22 may pass through the first light filter 52a within the bandpass range, such that the associated light from the fluorescent emission 32 is captured and identified by the camera controller 46. Similarly, the visible light emission 38 and the corresponding light reflected from the surgical site 26 may pass through a second light filter 52b, which may be configured to pass wavelengths of light in a visible range (e.g., 400 nm-650 nm). In this way, the camera system 10 may actively detect the fluorescent emission 32 and generate overlays, graphics, or other visual enhancements to augment the image data illuminated by the visible light emission 38 in the field of view 44.

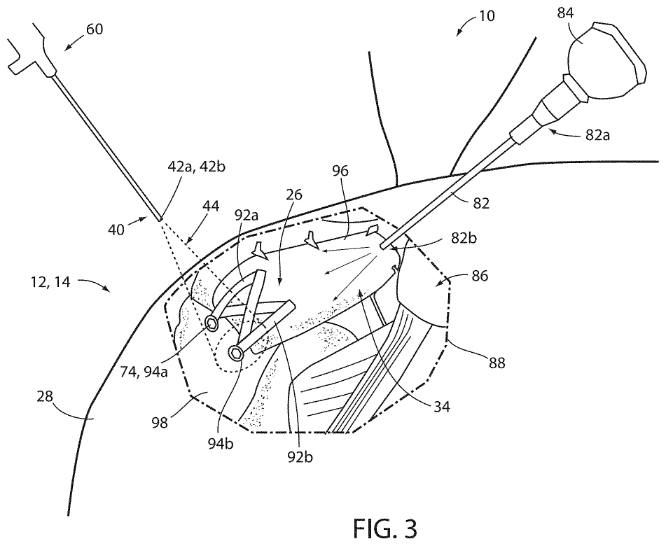

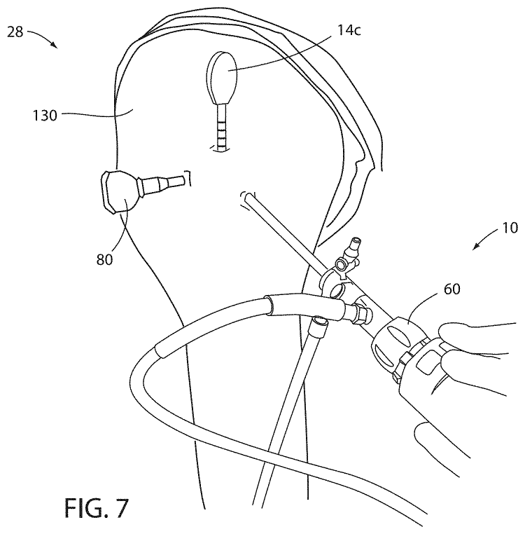

In addition to the first and second light filters 52a, 52b, the camera system 10 may further comprise additional filters, which may include one or more dichroic filters or mirrors configured to separate the fluorescent emission 32 from the visible light emission 38. Such filters, generally referred to as light filters 52, may be incorporated in an endoscope or camera 60, which may comprise the image sensors 42, light sources 36,40, and camera controller 46, as well as the light filters 52 in a unified package. For example, the camera 60 may comprise each of the light sources 36, 40, image sensors 42, filters 52, and the camera controller 46 in a compact endoscope similar to that discussed later in reference to FIGS. 3, 7, etc. In this way, the camera system 10 may be implemented in an easily manipulated package well suited for operation in the surgical environment 12. Though ICG is discussed in various examples of the disclosure, other fluorescents including methylene blue (MB), fluorescence, and protoporphyrin IX [PpIX], may be similarly implemented with the camera system 10.

With the image data associated with the visible light emission 38 detected independently of the fluorescent emission 32, the camera system 10 may provide for the enhancement of the fluorescent portions 22 in the image data. In this way, one or more colors, patterns, or other visual enhancements or overlays 62 may be superimposed or overlaid on the image data to generate enhanced image data for presentation on the display device 24. As shown in FIG. 2C, the location of the fluorescent portion 22 in the image data is emphasized by the overlay 62, such that the fluorescent portion 22 is clearly distinguishable from the remainder of the surgical implement 14 as well as the local environment in the surgical site 26. As discussed in further detail throughout the application, the enhanced image data may be implemented in a variety of ways to provide improved visualization of the surgical site 26 to assist in the identification of a presence, position, orientation, and/or dimension of various surgical implements 14.

Referring generally to FIGS. 1, 2A, 2B, and 2C; implementations and operating aspects of the camera system 10 are described in further detail. In general, ICG, fluorescein, PpIX, and methylene blue may correspond to dyes used in medical diagnostics. ICG has very low toxicity and high absorptance in a wavelength range of from about 600 nm to about 900 nm and a peak absorptance at about 780 nm. ICG emits fluorescence at a wavelength of about 830 nm. Additionally, fluorescent agents, such as ICG, that emit near-infrared radiation may be detectable through biological tissue. As used herein the terms “radiation” and “light” are used interchangeably. Another example of a fluorescent agent, PpIX may be excited over a blue color range (e.g., 405 nm) with a corresponding peak fluorescence of approximately 635 nm. MB is excited over a red-NIR color range (e.g., 600 nm) with a corresponding peak fluorescence of approximately 650 nm. Fluorescein has a peak absorption of approximately 490 nm with a fluorescent emission of approximately 520 nm. The gap between the absorption range and the emission range of each of the fluorescent agents is referred to as a Stokes shift, which may be utilized to distinguish between wavelengths associated with the excitation emission 34 and the resulting fluorescent emission 32.

In various examples, the fluorescent agent may be coated or used as an integral portion (e.g., embedded in a material or structure) of a surgical implement 14. In some cases, the fluorescent agent may be incorporated in the fluorescent portion 22 of the surgical implement 14 during manufacture. For example, a plastic surgical implement may have a fluorescent dye mixed into the plastic during manufacture. Additionally, light blocking packaging may be used to protect the fluorescent dye from light until the surgical implement 14 is ready for use. The surgical implement 14, such as, for example and without limitation, a sponge, a suture, a pin, a screw, a plate, a surgical tool, or an implant may be painted with a fluorescent material. As used herein, the term “surgical tool”, may comprise, without limitation, a biter, grasper, retriever, pick, punch, hook, probe, elevator, retractor or scissors. The surgical implement 14 may have a fluorescent agent coated on a portion to indicate a location, position, depth, orientation, or other characteristic of the surgical implement. Accordingly, the fluorescent portion 22 of the surgical implement 14 may be readily identified or detected in the enhanced image data provided by the camera system 10.

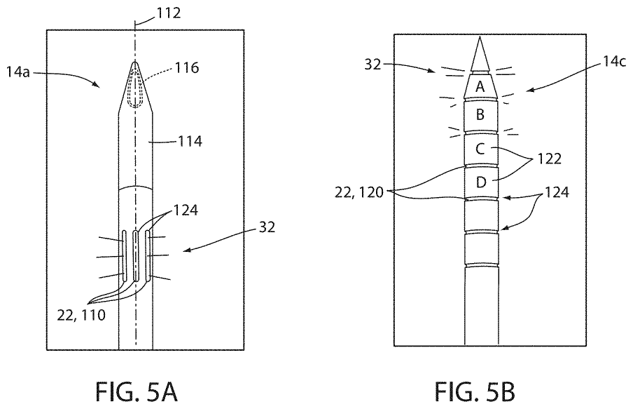

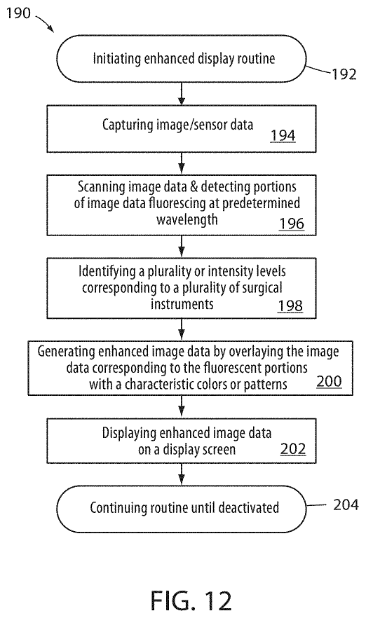

As discussed later in specific reference to FIGS. 5A and 5B, the fluorescent agent may incorporated in various fluorescent portions 22 of surgical implements 14 in patterns, shapes, and/or alphanumeric characters to identify the surgical implement 14 or to indicate dimensions, orientations, or proportions of implements 14 represented in the image data. The presence of a fluorescent agent in the surgical implement 14 may also enable surgeons to quickly check to make sure that no portion of a surgical implement 14 has been left in a surgical site 26. In some cases, the display controller 20 may be configured to process the image data associated with the fluorescent emission 32 (e.g., corresponding pixels in the field of view 44) to identify or classify one or more surgical implements 14 in the surgical site 26. For example, the display controller 20 may be configured to process a characteristic shape of the surgical implement 14 or one or more symbols represented in the image data captured by the first image sensor 42a (e.g., in the NIR range) to identify a type or category of the implement 14 based on a computer vision template. Such identification is discussed further in reference to FIG. 12.

In various implementations, the fluorescent agent in the surgical implement 14 may be excited using a light source that emits excitation light in the excitation wavelength range of the particular fluorescent agent. For example, when ICG is used as the fluorescent agent, ICG fluorescence may be excited using light in a wavelength range of from about 600 nm to about 900 nm and in some cases around 780 nm. In such cases, the light source 36 may be a light emitting diode or a laser emitting diode with a center frequency within or centrally within the excitation range of the ICG. The image sensors 42 may be, for example, a complementary metal-oxide-semiconductor (CMOS) sensor or a charge-coupled device (CCD) sensor. The camera 60 may also include optics, such as, for example, lenses, filters, mirrors, and prisms to direct and independently detect the wavelengths of light associated with the visible light source 40 and the fluorescent emission 32.

In some implementations, the camera 60 is implemented as an endoscopic camera, which may include the image sensors 42, light sources 36, 40, as well as the light filters 52. Accordingly, the camera 60 may include both the first light source 36 as an excitation light source for exciting the fluorescent agent and the second light source 40 in the form of a white light source for illuminating the surgical site 26 in the visible range of wavelengths. The camera 60 may further include a corresponding image sensor 42a or detector for detecting the fluorescent emission 32 and an image sensor 42b or detector for detecting and recording image data in the visible light range. In some cases, the camera 60 may have additional light sources for exciting multiple fluorescent agents or for detecting other non-visible attributes of a surgical field. An example of a camera system usable to detect fluorescent agents in surgical implements is the Arthrex Synergy IDTM camera system which has a camera head and a camera control unit. The Arthrex Synergy IDTM camera system has a light source for exciting fluorescence from ICG and is capable of detecting visible and near infra-red (NIR) light such as light emitted by ICG.

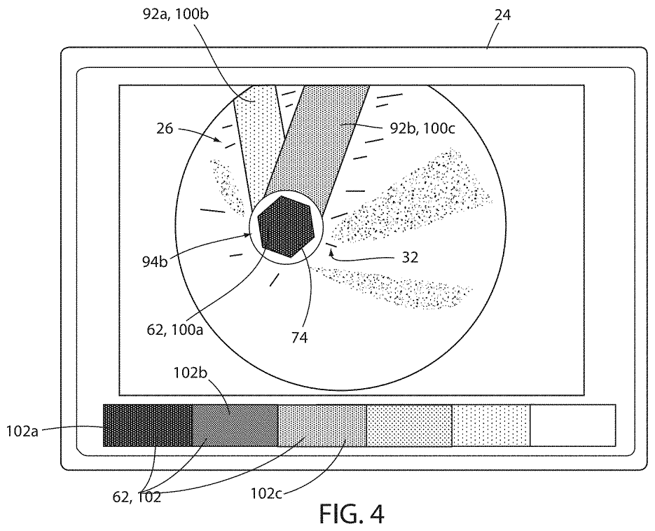

Referring again to FIG. 1, exemplary enhanced image data 70 is demonstrated on the display device 24. In the example, an acting end or distal end of the shaver 14a is shown demonstrating a first fluorescent portion 22a including a directional orientation marker 72. A similar example of the surgical implement 14 in the form of a shaver 14a is shown with improved detail in FIG. 4A. As shown the orientation marker 72 may be overlaid on the visible image data to provide a clear indication of the relative orientation of the shaver 14a in the surgical site 26. Though the orientation marker 72 may seem trivial in cases where the surgical implement 14 is clearly visible in the image data, the overlay 62 aligned with the fluorescent emission 32 demonstrated in the enhanced image data may provide a clear indication of the orientation and/or position of the surgical implement 14 even in cases where a cavity of the surgical site is obstructed or clouded by particles, blood, tissue debris, etc.

Additionally, FIG. 1 demonstrates an example of the surgical implement 14 in the form of an anchor 14b. In various cases, an anchor or various surgical implants may become over grown by tissue, calcium, or other substances that may mask them from visibility from the visible light emission 38 and the corresponding second image sensor 42b. As shown, a colored overlay 62 is generated by the display controller 20 in a portion of the image data associated with a second fluorescent portion 22b. The overlaid or superimposed color may highlight a portion of the anchor 14b, such that the location of a hexalobe or drive head 74 is visible in the enhanced image data. In cases where the drive head 74 is masked behind biological tissue, the excitation emission 34 and the resulting fluorescent emission 32 may penetrate the tissue such that the display controller 20 may detect the fluorescent portion 22 and demonstrate the location of the head 74 in the enhanced image data.

Referring now to FIGS. 3 and 4, an example of an application of the camera system 10 is described in reference to an exemplary shoulder repair operation. As depicted, the camera 60 is implemented as an endoscope that incorporates the second light source 40 configured to output the visible light emission 38 within the field of view 44 of the image sensors 42a, 42b. As shown in FIG. 3, the first light source 36 associated with the excitation emission 34 may be incorporated in a dedicated lighting device 80. The lighting device 80 may comprise an elongated shaft 82 extending between a proximal end portion 82a and a distal end portion 82b. The excitation emission 34 may be output from the first light source 36 via the distal end portion 82b of the elongated shaft 82. A control circuit and power supply may be enclosed in a housing 84 in connection with the proximal end portion 82a. In this configuration, the excitation emission 34 may originate from a different origin than the field of view 44. The dedicated lighting device 80 may project the excitation emission 34 into various portions or regions of the surgical site 26 without having to maneuver the camera 60. Accordingly, implementations of the camera system 10 incorporating the dedicated lighting device 80 separate from the camera 60 may provide for independent illumination of the various regions within the surgical site 26 without maneuvering the camera 60 or independent of the position of the camera 60.

Though discussed in reference to the excitation emission 34 being output from the dedicated lighting device 80, either or both of the light sources 36, 40 may be implemented in the dedicated lighting device 80 to output light in various ranges of wavelengths. In some implementations, the lighting device 80 or the camera 60 may be configured to emit a beam of light with a diameter small enough for targeting items in the surgical field for further action by a surgeon. In an implementation, the beam diameter may be less than about 5 mm. In some cases, the beam diameter may be less than about 2 mm or than about 1 mm. In general, the lighting device 80 or camera 60 may be configured to emit a beam of light of sufficient brightness and density to be detected within a surgical field. For example, in some cases, high sensitivity sensors 42 have been measured to detect light at intensities of 10 nW/cm² or less (e.g., a high sensitivity CMOS sensor). The light sources 36, 40 may be positioned proximal to a distal end of the light emitting device 80 or camera 60. Additionally, the light source 36, 40 may be positioned away from the distal end and light emitting device 80 or camera 60 from the light source communicated to the distal end such as by, for example, fiber optics. The light emitted by the light emitting device 80 and/or camera 60 may have a variable shape that may be adjusted, such as by using optics to allow a user to better illuminate a desired target.

In some implementations, one or both of the light sources 36, 40 may be incorporated into a surgical instrument 14 other than the endoscopic camera system 10, for example, in a probe, a shaver 14a, an ablation device, or other instrument. In some examples, an LED may be located at a distal end of the device or instrument. In some example, a probe or other device may be formed at least partially of a light pipe that may receive light from an LED, laser, or other light source external to the body and transmit the radiation to the distal end of the instrument. The light emitting device 80 may be powered by an isolated power source coupled to the light emitting device. Additionally, the light emitting device 80 may be battery powered. The battery powered light emitting device may be configured for a single use or may be configured with a rechargeable battery for multiple uses. The light emitting device 80 may be packaged in a sterile container for a single use. Additionally, the light emitting device 80 may be configured for sterilization and repeated use. The light emitting device 80 may be a rigid device or a flexible device. The light emitting device may be an articulatable device.

Additionally, the light emitting device 80 or light sources 36, 40 may be placed outside of a surgical field or site 26 and light directed through biological tissue for detection by the camera 60 positioned in the surgical field. Additionally, the light emitting device may direct light from a surgical field through tissue for detection by a device positioned outside of a surgical field. In some cases, the light emitting device 80 may be placed outside of a body and direct light through tissue for detection by the camera 60 positioned inside the body. Additionally, the light emitting device 80 may be placed inside of a body and direct light through tissue for detection by a camera (e.g., the camera 60) positioned outside of the body. Additionally, the light emitting device 80 may be placed in a first portion of a surgical site 26 and direct light through tissue for detection in a second portion of the surgical site 26.

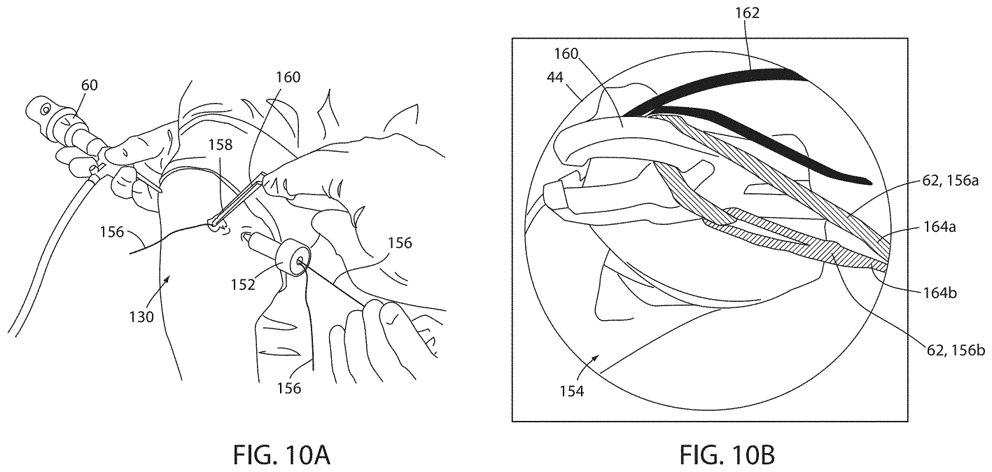

As demonstrated in FIG. 3, a shoulder cavity 86 is revealed via a cutaway section 88. However, in a typical arthroscopic procedure, the shoulder cavity 86 would be enclosed, such that the internal anatomy of the patient 28 would not be visible as depicted in FIG. 3. To accurately visualize the shoulder operation corresponding to FIG. 3, the distal end of the camera 60 and the dedicated light source 80 would protrude through the outer tissue and into the shoulder cavity 86, similar to the examples demonstrated in FIGS. 7 and 10A, as later discussed. According, the cutaway section 88 in FIG. 3 may provide for a simplified representation of an arthroscopic procedure to demonstrate the internal anatomy and may similarly be representative of an open surgery where the camera 60 and dedicated lighting device 80 may be positioned outside and provide illumination into the shoulder cavity 86.

Referring now to FIGS. 3 and 4, a plurality of sutures 92a, 92b and anchors 94a, 94b are shown implemented in connection with a shoulder tendon 96 and humorous 98 of the patient 28. As shown, the sutures 92 may comprise a first suture 92a and a second suture 92b. The first suture 92a is in connection with a first anchor 94a that connects the first suture to the humorous 98. The second suture 92b is in connection with the humorous 98 via a second anchor 94b. Though clearly represented in FIG. 3, a view of the surgical site 26 may be clouded by blood and particulates within the shoulder cavity 86. Accordingly, the view and relative orientation of the camera 60 in relation to the surgical site 26 may not be readily apparent from the image data demonstrated on the display device 24.

In addition to the obstructions in the field of view 44 within the shoulder cavity 44, in some cases, an anchor (represented in FIG. 4 as the second anchor 94b) may be masked or hidden beneath tissue or overgrowth. In such cases, the second anchor 94b may be nearly completely hidden from view and challenging to detect within the image data captured by the camera 60. In order to improve the visibility of the second anchor 94b, a fluorescent agent may be incorporated in a portion of the second anchor 94b, exemplified as a first fluorescent portion 100a (See, FIG. 4) incorporated in a drive head 74 or hexalobe. In addition to the first fluorescent portion incorporated in the second anchor 94b, each of the first suture 92a and the second suture 92b may also include corresponding second and third fluorescent portions 100b and 100c. Each of the fluorescent portions 100a, 100b, and 100c may be illuminated by the excitation emission 34 output, in this example, from the first light source 36 of the dedicated lighting device 80. In response to receiving the excitation emission 34, each of the fluorescent portions 100a, 100b, 100c may become excited to output corresponding fluorescent emissions 32.

In some cases, the fluorescent emissions 32 output from the fluorescent portions 100a, 100b, 100c may vary in wavelengths due to different compositions or combinations of fluorescent agents incorporated therein. In other cases, a concentration of a common fluorescent agent (e.g., ICG dye) may be incorporated at different levels in each of the fluorescent portions 100a, 100b, 100c. Accordingly, in response to receiving the excitation emission 34, each of the fluorescent emissions 32 output from the fluorescent portions 100a, 100b, 100c may vary in wavelength or intensity based on the composition of fluorescent agents or concentration of fluorescent agents incorporated therein. Based on the variations in the intensity or wavelengths associated with the fluorescent emissions 32, the display controller 20 may be operable to distinguish among the different fluorescent portions 100a, 100b, 100c and overlay each of the fluorescent portions 100a, 100b, 100c with different characteristic colors 102. Accordingly, the camera system 10 may be configured to distinguish among a plurality of fluorescent portions 100a, 100b, 100c and assign different respective characteristic colors 102 or patterns, such that the enhanced image data demonstrated on the display device 24 clearly distinguishes the locations of each of the surgical implements 14 (e.g., 92a, 92b, and 94b).

To be clear, the sutures 92a, 92b and second anchor 94b demonstrated in FIG. 4 may appear in the image data as being dull and nearly indistinguishable from their surroundings when viewed solely via the visible light emission 38. However, based on the overlays 62 applied by the display controller 20 over the corresponding fluorescent portions 100a, 100b, 100c; the enhanced image data may clearly differentiate each of the surgical implements 14 based on a corresponding characteristic color 102a, 102b, 102c or pseudo-color overlaid on the image data associated with the visible light emission 38. As shown, the characteristic colors 102 may include a first color 102a, a second color 102b, and a third color 102c. The first color 102a may be incorporated on the first fluorescent portion 100a coating the drive head 74 or hexalobe of the second anchor 94b. The second color 102b and the third color 102c may be incorporated within a constituent material forming the first suture 92a and the second suture 92b, respectively. Each of the characteristic colors 102 may be visually distinguishable based on a predetermined display configuration stored within the display controller 20.

In some cases, the characteristic colors 102 or patterns associated with the enhanced image data may be customized or modified to suit the preferences of a specific user. For example, some users may prefer a wide range of colors to assist in distinguishing among the various surgical implements 14, while others may prefer subtle color differences that may not distract their view from other aspects within the surgical site 26. In some cases, the display controller 20 may adjust a color template or color configuration of the characteristic colors 102 or patterns based on the colors of the local environment demonstrated in the image data captured by the second image sensor 42 associated with the visible light emission 38. For example, if the image data illuminated by the visible light emission 38 is displayed primarily in warm hues (e.g., red, yellow, orange), the display controller 20 may assign a cool color template (e.g., blue, purple, green) to distinguish the fluorescent portions 100a, 100b, 100c from the remainder of the image data in the field of view 44. Similarly, if the image data is dark, light or contrasting hues or patterns may be automatically applied to contrast the image data. Accordingly, the camera system 10 may provide for a variety of formats and color templates associated with the enhanced image data to assist in the visualization of the surgical site 26.

Referring to FIGS. 5A and 5B, exemplary surgical implements 14 are shown comprising fluorescent portions 22 configured to assist a user in a recognition of an orientation or position of the surgical implements 14 as represented in the enhanced image data generated by the camera system 10. As shown in FIG. 5A, an acting end of the shaver 14a is shown demonstrating a plurality of longitudinal markings 110 formed by the fluorescent portions 22. The longitudinal markings may extend along a longitudinal axis 112 of the shaver 14a and be evenly spaced radially about an elongated body 114. A shaver head 116 is demonstrated in phantom opposing the face pictured in FIG. 5A. In this configuration, the longitudinal markings 110 comprising the fluorescent portions 22 may be illuminated to output the fluorescent emission 32 in response to the excitation emission 34, such that the enhanced image data may demonstrate an orientation of the surgical implement 14 or shaver 14a in relation to an actuator direction (e.g., direction of the shaver head 116).

Referring to FIG. 5B, the surgical implement 14 is demonstrated as an exemplary needle or probe 14c shown comprising a plurality of lateral markings 120 corresponding to the fluorescent portions 22. As shown, the lateral markings 120 are implemented as a plurality of graduated segments demonstrating a scale associated with a position of the surgical implement 14 or probe 14c. Similar to the longitudinal markings 110, the lateral markings 120 may incorporate the fluorescent agent in the fluorescent portions 22 and output the fluorescent emission 32 in response to receiving the excitation emission 34. In addition to the lateral markings 120, the probe 14c may include one or more characters 122 or symbols, which may also incorporate fluorescent dyes or agents, such that the characters 122 may be overlaid in the image data to emphasize the associated symbols in the image data. The longitudinal markings 110 and lateral markings 120 may be implemented in various combinations to assist an operator of the associated surgical implements 14 to identify an orientation, position, and/or relative measurement of the surgical implement 14 as presented in the enhanced image data on the display device 24.

In some cases, the longitudinal markings 110, lateral markings 120, or various additional fluorescent portions 22 incorporated on the surgical implements 14 may be disposed within a groove 124 or indentation formed in an exterior surface of the surgical implement 14. By including the fluorescent portions 22 in the grooves or indentations associated with the orientation or positional markings 110, 120; the resulting fluorescent emissions 32 output from the grooves 124 or indentations may be captured in the field of view 44 of the camera system 10 through an orientation aperture associated with an interior surface of each of the grooves 124 directed to or facing the corresponding image sensors 42a, 42b of the camera 60. In this configuration, the dimensional or orientational markings 110, 120 incorporated on the surgical implement 14 may be hidden from the field of view 44 of the camera 60 until a portion of the fluorescent emission 32 is output from the corresponding fluorescent portions 22 disposed in the grooves 124. The result of the fluorescent portions 22 disposed in the grooves 124 may be an improved accuracy achieved similar to a sight that only exposes the fluorescent emission 32 when an interior surface of each of the grooves 124 is visible through the corresponding orientation aperture. In this way, the dimensional and orientational features (e.g., 110, 120) of the surgical implements 14 may provide for improved accuracy in determining the relative positioning or orientation of the surgical implement 14.

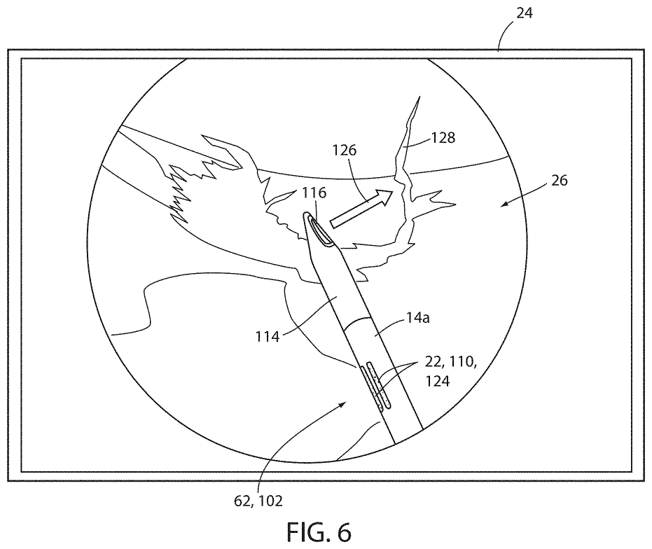

Referring now to FIG. 6, the exemplary shaver 14a is shown in the field of view 44 of the camera 60 demonstrating enhanced image data including overlays 62 of characteristic colors 102 over the longitudinal markings 110 formed by the grooves 124 and the fluorescent portions 22. As shown, the longitudinal markings 110 may assist an operator in identifying a direction of the shaver head 116 demonstrated by the arrow 126. For example, as a result of seeing two of the three longitudinal markings 110 on the display device 24, a user of the shaver 14a may visually identify, from the longitudinal markings 110 enhanced by the overlay 62, that the shaver head 116 is directed toward an opposite side of the longitudinal markings 110. As shown, the longitudinal markings 110 are positioned on a left-facing side of the shaver 14a, such that the operator may recognize that the shaver head 116 is directed toward a right side represented on the display device 24. Such indications of the orientation of the surgical implement 14 may be particularly beneficial in cases where the shaver head 116 is hidden behind tissue 128 or debris in the field of view 44. Accordingly, the longitudinal markings 110 may assist a user in determining the relative orientation of the surgical implement 14.

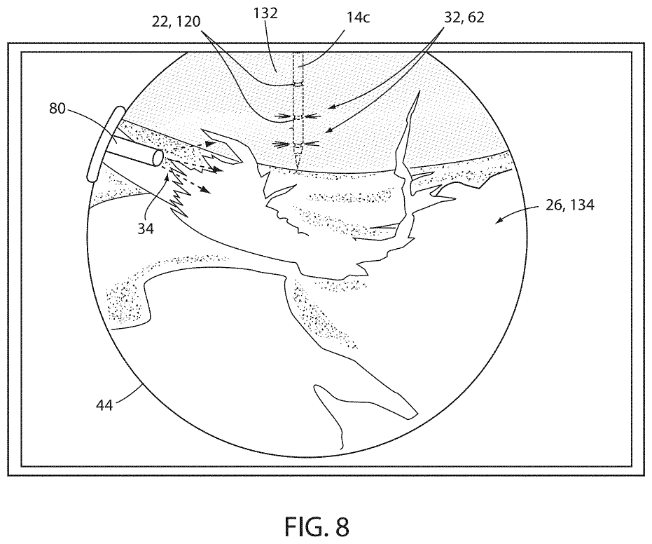

Referring now to FIG. 7, an additional exemplary illustration of an arthroscopic procedure on a shoulder 130 of the patient 28 is shown. FIG. 8 demonstrates enhanced image data associated with the field of view 44 captured by the camera 60 positioned as depicted in FIG. 7. As demonstrated in FIGS. 7 and 8, the probe 14c is demonstrated penetrating biological tissue 132 within a shoulder cavity 134. As previously discussed, the excitation emission 34 may be output from the first light source 36 incorporated in the dedicated lighting device 80. The excitation emission 34 may be transmitted within the cavity 134 and penetrate through the biological tissue 132 (e.g., cartilage, muscle, tendons, bone, etc.) to impinge upon the fluorescent portions 22 formed by the lateral markings 120. In response to receiving the excitation emission 34, the fluorescent agent incorporated in the fluorescent portions 22 of the lateral markings 120 may output the fluorescent emission 32. The light energy emitted from the fluorescent portions 22 may also be transmitted through the biological tissue 132 and into the cavity 134, such that the near-infrared image sensor 42a may capture the fluorescent emissions 32 in the field of view 44.

In response to detecting the fluorescent emission 32 in the image data captured by the first image sensor 42a, the display controller 20 of the camera system 10 may overlay the pixels in the image data associated with the fluorescent emission 32 with the overlay 62 (e.g., characteristic colors 102 or patterns) to generate the enhanced image data. Accordingly, the camera system 10 may provide for the detection and tracking of the position of one or more surgical implements 14 through biological tissue 132 by detecting the fluorescent emission 32. Once detected, the display controller 20 may further overlay, mark, or enhance corresponding portions of the image data to demonstrate the surgical implements 14 that would otherwise be completely hidden from a conventional camera system.

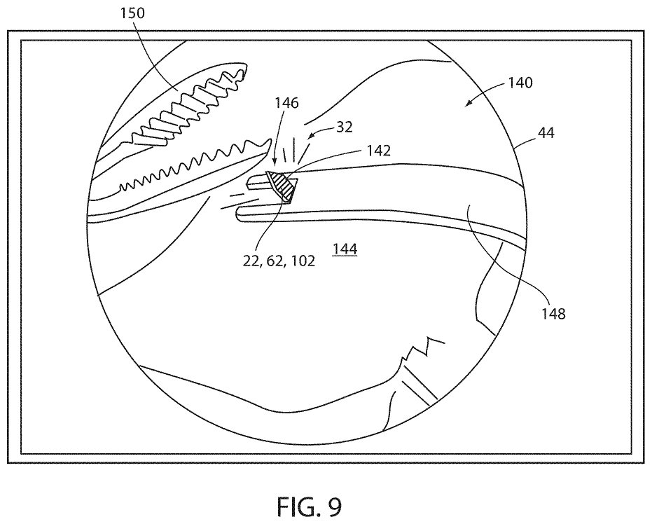

Referring now to FIG. 9, an exemplary surgical cavity 140 is shown demonstrating a distal tip of a probe or needle 142 beginning to protrude through biological tissue 144. As depicted in the enhanced image data demonstrated on the display device 24 of the camera system 10, a distal tip 146 of the needle 142 is overlaid by a characteristic pattern or color 102. Similar to other examples, the characteristic pattern or color 102 overlaid on the distal tip 146 of the needle 142 may be detected by the display controller 24 in response to the corresponding presence of the fluorescent emission 32 in the image data captured by the combined image sensors 42a, 42b. In the example provided, the distal tip 146 of the needle 142 may be introduced blindly into the surgical cavity 140. Accordingly, it may be challenging for a surgeon or physician to accurately determine a position of a depressing instrument 148 and grasper 150 to effectively guide and interact with the distal tip 146. However, due to the incorporation of the fluorescent portion 22 on the distal tip 146, the fluorescent emission 32 may penetrate the biological tissue 144 and be detected by the display controller 20 before the distal tip 146 begins to protrude through the biological tissue 144. For example, in response to identifying the fluorescent emission 32 in the field of view 44, the display controller 20 may enhance the corresponding portion of the image data associated with the fluorescent emission 32 with the overlay 62. In this way, a surgeon may identify a location of the biological tissue 144 through which the distal tip 146 of the needle 142 will protrude prior to the distal tip 146 breaching the surface of the biological tissue 144. In this way, the enhanced image data provided by the camera system 10 may improve the accuracy associated with an operation by displaying a location of a surgical implement that would otherwise be invisible in a visible light range captured by the second imager or visible light image sensor 42b.

In some examples, the excitation light source or first light source 36 may output the excitation emission 34 at an intensity sufficient to penetrate biological tissue as discussed herein. For example, the first light source 36 may output the excitation emission 34 at an intensity ranging from approximately 1 mW/cm² to 1 W/cm². In some cases, the light intensity may be higher or lower depending on the specific light emitter technology implemented and the application. Depending on the application and the duration over which the excitation emission 34 is to be activated, the intensity of the excitation emission 34 may be limited or pulsed to control excess heat generation and limit damage to the biological tissue. As previously discussed, the excitation emission 34 may comprise wavelengths of radiation ranging from approximately 650 nm to 900 nm in the near-infrared range. For reference, the visible light emission 38 associated with the second light source 40 may be output in wavelengths corresponding to visible colors of light associated with the acuity of a human eye ranging from 400 nm to approximately 650 nm. The penetration of the excitation emission 34 and/or the fluorescent emission 32 through biological tissue may extend approximately from a depth of 1 mm to depths or thicknesses of biological tissue exceeding 10 mm. Experimental results have demonstrated a loss of intensity of emissions similar to the excitation emission 34 and the fluorescent emission 32 in the near-infrared range at a rate of approximately 3%-10%/mm of biological tissue penetrated. Accordingly, the first image sensor 42a may detect the fluorescent emission 32 or the excitation emission 34 after the corresponding light energy has penetrated multiple millimeters of biological tissue. Therefore, the camera system 10 may identify the relative location or orientation of the various surgical implements 14 and demonstrate the locations in the enhanced image data in a variety of cases where the surgical implements 14 may be hidden behind layers of biological tissue having various thicknesses.

Referring now to FIGS. 10A and 10B, yet another exemplary application of the surgical camera system 10 is shown demonstrating an arthroscopic shoulder repair of the patient 28. As demonstrated in FIG. 9, an anterior cannula 152 provides access into a surgical cavity 154 to manipulate a plurality of sutures 156a, 156b. In operation, a surgeon may access the surgical cavity 154 via a skid 158. In order to reach the sutures 156 within the surgical cavity 154, a grasper 160 may be implemented to selectively engage one of the sutures 156. As demonstrated in FIG. 10B, the field of view 44 of the camera 60 demonstrates an arthroscopic view of a first suture 156a, second suture 156b, and a lasso 162 that may further be implemented to manipulate and loop the sutures 156. Even with extensive knowledge of the procedures and associated visible colors incorporated on the sutures 156, surgeons and physicians still may have difficulty distinguishing the first suture 156a from the second suture 156b. Distinguishing the sutures 156 may become particularly challenging when the fluid within the surgical cavity 154 is encumbered by debris or blood that may further mask any defining features of the sutures 156.

As previously discussed in reference to FIGS. 3 and 4, the first suture 156a may include a first concentration of the fluorescent agent and the second suture 156b may include a second concentration of the fluorescent agent. Accordingly, in response to receiving the excitation emission 34, each of sutures 156a, 156b may output different intensities of the fluorescent emission 32. These intensities of the fluorescent emission 32 may be identified and distinguished by the display controller 20 based on the image data in the near-infrared range captured by the first image sensor 42a. In response to the differing intensities of the fluorescent emissions 32, the display controller 20 may overlay each of the sutures 156a, 156b with different characteristic patterns 164a, 164b as demonstrated by FIG. 10B. In this way, the display controller 20 may identify the fluorescent emissions 32 at various intensities to distinguish among a plurality of surgical implements 14 identified in the field of view 44 of the camera system 10. The overlays 62, shown as characteristic patterns, of the sutures 156a, 156b may similarly be implemented as characteristic colors or markers (e.g., notification windows, superimposed graphics, etc.) to assist in identifying and distinguishing among surgical implements 14 depicted in the image data of the camera system 10.

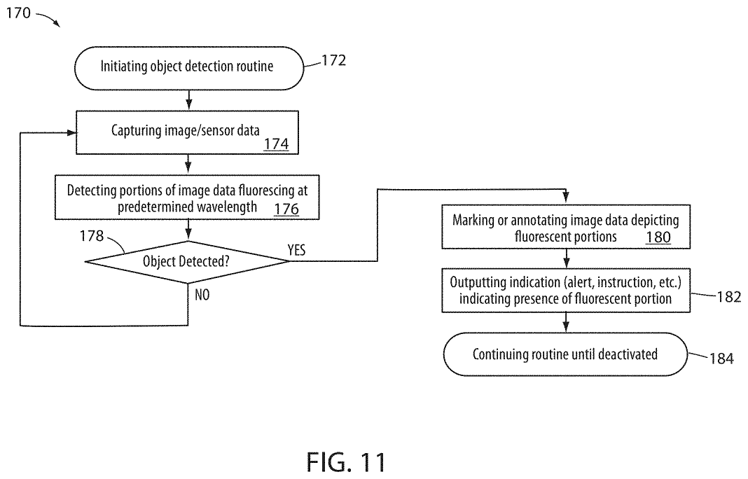

Referring now to FIG. 11, an exemplary flowchart is shown demonstrating a method for detecting an object with the camera system 10 as discussed herein. The method 170 may begin in response to an activation of the camera system 10 or initiation of an object detection routine 172. As discussed in various examples, the camera 60 may be controlled by the camera controller 46 to capture image or sensor data via one or more of the image sensors 42a, 42b (174). Once the image data is captured by the image sensors 42a, 42b, the display controller 20 may detect one or more portions of the image data or pixels within the field of view 44 that include wavelengths of light corresponding to the fluorescent emission 32 from the fluorescent portions 22 (176). Based on the image data processed by the display controller 20, the method 170 may continue in step 178 to determine if one or more surgical implements 14 are detected in response to the presence of the fluorescent emission 32. If no implements 14 are detected in step 178, the method 170 may return to step 174 to continue capturing the image or sensor data and processing the image data to identify the fluorescent emission 32 in steps 174 and 176.

In step 178, if an object associated with the fluorescent emission 32 is detected in the image data, the method 170 may continue to mark, overlay, or annotate the image data to emphasize the regions in the field of view 44 where the fluorescent emission 32 is detected (180). The marked or annotated image data generated in step 180 may correspond to the enhanced image data comprising one or more overlays 62 in the form of characteristic colors, patterns, or other indicating features that may assist a viewer in recognizing a location, orientation, dimensions, proportions, or other information related to the surgical implement 14 from which the fluorescent emission 32 was emitted and detected by the camera system 10. Examples of surgical implements may include a biter, grasper, retriever, pick, punch, hook, probe, elevator, retractor or scissors. In some cases, the surgical implements 14 may correspond to items configured to trigger an alert or notification of the camera system 10 to indicate the detection of their presence. For example, partial components of tools, implants, sponges, or other various surgical implements within the surgical site 26 may be detected by camera system 10 in response to the presence of the fluorescent emission 32. In response to such a detection, the method 170 may output an indication (e.g., an alert, instruction, notification, etc.) indicating the presence of a fluorescent portion 22 and alerting a surgeon or medical professional of the presence of the corresponding surgical implement 14 (182). In some cases, the programming of the camera system 10 may define specific surgical implements 14 that may be associated with the fluorescent emission 32. In such cases, the notification output in step 182 may indicate the specific type or category of the surgical implement 14 identified in the image data by the camera system 10. Following step 182, the detection routine may continue until it is deactivated by an operator, as demonstrated in step 184.

Referring now to FIG. 12, a flowchart is shown demonstrating a method 190 for displaying enhanced image data in accordance with the disclosure. The method 190 may begin in response to the initiation of an enhanced image data display routine by the camera system 10 (192). Once initiated, the method 190 may continue to step 194 to capture image or sensor data with the image sensors 42a and 42b. Once captured, the display controller 20 may scan the image data and detect portions of the image data with wavelengths corresponding to the fluorescent emission 32 as detected by the first image sensor 42a (196). In some cases, the method 190 may identify a plurality of fluorescent emissions 32 depicted in the image data at a plurality of intensity levels corresponding to a plurality of fluorescent portions 22 that may include varying concentrations of fluorescent agents (198). As previously discussed, each of the fluorescent portions 22 of the surgical implements 14 detected in the field of view 44 may include a distinctive concentration of the fluorescent agent, such that the resulting fluorescent emissions 32 may be output and detected by the first image sensor 42a at different intensity levels. Based on the different intensity levels, the display controller 20 may assign the overlays 62 as different characteristic colors in the image data to generate the enhanced image data for display on the display device 24 (200).

In some cases, the display controller 20 may identify different intensities of the fluorescent emission 32 over time, such that the characteristic colors or patterns associated with the overlay 62 of the enhanced image data may be maintained even in cases where the corresponding surgical implements 14 are not simultaneously presented in the image data. For example, the display controller 20 may be preconfigured to associate a lower intensity fluorescent emission 32 with a first color, a medium intensity fluorescent emission 32 with a second color, and a third intensity fluorescent emission 32 with a third color. The relative intensities may correspond to percentages or relative levels of luminance associated with each of the fluorescent emissions 32. For example, if three levels of luminance are detected, a maximum intensity may be associated with the third color. An intermediate intensity may be associated with the second color, and a minimum or lowest intensity may be associated with the first color. Once the enhanced image data is generated, it may further be selectively displayed on the display device 24 by controlling an interface of the display controller (202). Following step 202, the display routine may continue until deactivated (204).

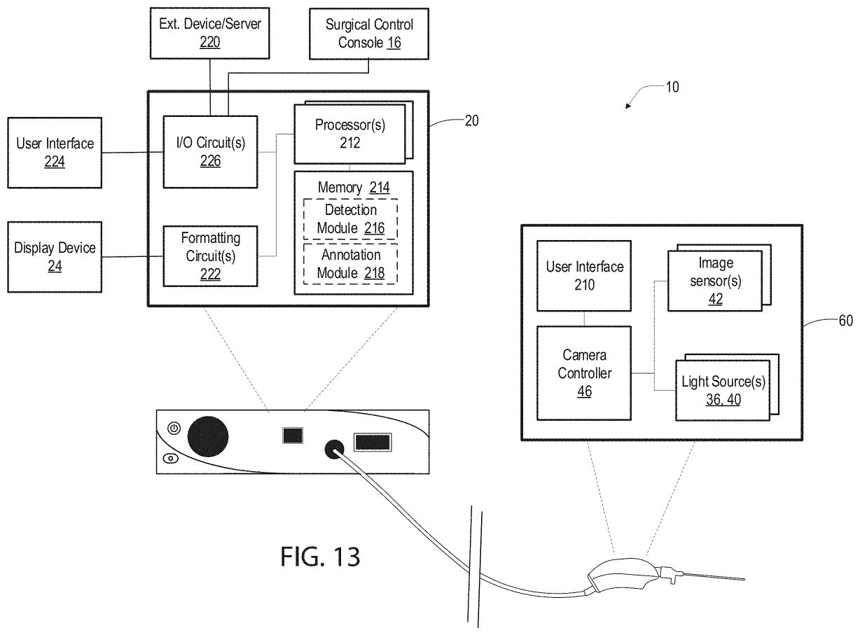

Referring now to FIG. 13, a block diagram of the camera system 10 is shown. As discussed throughout the disclosure, the system 10 may comprise a camera 60 in communication with a display controller 20. The camera 60 may comprise a plurality of light sources 36, 40; at least one image sensor 42 (e.g., 42a, 42b); a camera controller 46; and a user interface 210. In various implementations, the camera 60 may correspond to an endoscope with an elongated scope comprising a narrow distal end suited to various non-invasive surgical techniques. For example, the distal end may include a diameter of less than 2 mm. As demonstrated, the camera 60 may be in communication with the display controller 20 via communication interface. Though shown connected via a conductive connection, the communication interface may correspond to a wireless communication interface operating via one or more wireless communication protocols (e.g., WiFi, 802.11 b/g/n, etc.).

The light sources 36, 40 may correspond various light emitters configured to generate light in the visible range and/or the near infrared range. In various implementations, the light sources 36, 40 may include light emitting diodes (LEDs), laser diodes, or other lighting technologies. As previously discussed, the first light source 36 may generally correspond to a laser emitter configured to output emissions in the near infrared range including wavelengths from approximately 650 nm to 900 nm. In some instances, the first light source 36 may output the excitation emission 34 ranging from 650 nm to 680 nm with a center frequency of approximately 670 nm. In some cases, the first light source 36 may output the excitation emission 34 in a range of wavelengths from approximately 740 nm to 780 nm. More generally, the wavelengths associated with the first light source 36 and the excitation emission 34 may be selected to effectively energize the fluorescent agent of the fluorescent portion 22. The second light source 40 may correspond to a white light source in the visible spectrum including wavelengths ranging from approximately 380 nm to 700 nm or from approximately 400 nm to 650 nm.

The image sensors 42a, 42b may correspond to various sensors and configurations comprising, for example, charge-coupled devices (CCD) sensors, complementary metal-oxide semiconductor (CMOS) sensors, or similar sensor technologies. As previously discussed, the system 10, particularly the display controller 20 may process or compare the image data captured by each of the image sensors 42 to identify the fluorescent emission 32 and apply the overlay 62 in the form of one or more colors (e.g., the characteristic colors 102), patterns, markers, graphics, messages, and/or annotations indicating the presence and/or location of the fluorescent emission 32 in the image data. In operation, the light filters 52a, 52b (e.g. bandpass filters) may filter and effectively separate the combined wavelengths of the fluorescent emission 32 and the visible light emission 38 in the field of view 44. Accordingly, the filtered light received by the first image sensor 42a may provide a map identifying locations of the fluorescent emission 32 and the corresponding locations of the fluorescent portions 22 of the surgical implements 14 in the image data.

The camera controller 46 may correspond to a control circuit configured to control the operation of image sensors 42a, 42b and the light sources 36, 40 to provide for the concurrent or simultaneous capture of the image data in the visible light spectrum as well as the near infrared spectrum or wavelength associated with the fluorescent emission 32. Additionally, the camera controller 46 may be in communication with a user interface 210, which may include one or more input devices, indicators, displays, etc. The user interface may provide for the control of the camera 60 including the activation of one or more routines as discussed herein. The camera controller 46 may be implemented by various forms of controller, microcontrollers, application-specific integrated controllers (ASICs), and/or various control circuits or combinations.

The display controller 20 may comprise a processor 212 and a memory 214. The processor 212 may include one or more digital processing devices including, for example, a central processing unit (CPU) with one or more processing cores, a graphics processing unit (GPU), digital signal processors (DSPs), field programmable gate arrays (FPGAs), application specific integrated circuits (ASICs) and the like. In some configurations multiple processing devices are combined into a System on a Chip (SoC) configuration while in other configurations the processing devices may correspond to discrete components. In operation, the processor 212 executes program instructions stored in the memory 214 to perform the operations described herein.

The memory 214 may comprise one or more data storage devices including, for example, magnetic or solid state drives and random access memory (RAM) devices that store digital data. The memory 214 may include one or more stored program instructions, object detection templates, image processing algorithms, etc. As shown, the memory 214 may comprise a detection module 216 and an annotation module 218. The detection module 216 include instructions to process the image data identifying the fluorescent emission 32 from the first image sensor 42a and detect the locations in the field of view 44 from which the fluorescent portion 22 of the surgical implement 14 emitted the fluorescent emission 32. In some cases, the detection module 216 may include instructions to detect or identify a type or classification associated with the surgical implement 14 in the image data captured by the camera 60. For example, the processor 212 may access instructions in the detection module 216 to perform various processing tasks on the image data including preprocessing, filtering, masking, cropping and various enhancement techniques to improve detection capability and efficiency. Additionally, the detection module 216 may provide instructions to process various feature detection tasks including template matching, character recognition, feature identification or matching, etc. In some examples, the detection module 216 may also include various trained models for object detection and/or labeling surgical implements 14 or related objects. In some implementations, the detection of a surgical implement, either by identity, presence, or classification, may initiate an instruction to output an alert or notification on the display device 24, the control console 16, an external device or server 220, or various connected devices associated with the surgical camera system 10.

The annotation module 218 may comprise instructions indicating various marking or overlay options to generate the enhanced image data as well as corresponding display filters to superimpose or apply the overlays 62 to the image data. As previously discussed, the enhanced image data may also include one or more graphics, annotations, labels, markers, and/or identifiers that indicate the location, presence, identity, or other information related to a classification or identification of the surgical implement 14. The annotation module 218 may further provide instructions to generate, graphics, labels, overlays or other associated graphical information that may be applied to the image data captured by the second image sensor 42b (e.g., the visible light sensor) to generate the enhanced image data for display on the display device 24.

The display controller 20 may further comprise one of more formatting circuits 222, which may process the image data received from the camera 60, communicate with the processor 212, and output the enhanced image data to the display device 24. The formatting circuits 222 may include one or more a signal processing circuit, analog to digital converters, digital to analog converters, etc. The display controller may comprise a user interface 224, which may be in the form of an integrated interface (e.g., a touchscreen, input buttons, an electronic display, etc.) or may be implemented by one or more connected input devices (e.g., a tablet) or peripheral devices (e.g., keyboard, mouse, etc.). As shown, the controller 20 is also in communication with an external device or server 220, which may correspond to a network, local or cloud-based server, device hub, central controller, or various devices that may be in communication with the display controller 20 and more generally the camera system 10 via one or more wired (e.g., Ethernet) or wireless communication (e.g., WiFi, 802.11 b/g/n, etc.) protocols. For example, the display controller 20 may receive updates to the various modules and routines as well as communicate sample image data from the camera 60 to a remote server for improved operation, diagnostics, and updates to the system 10. The user interface 224, the external server 220, and/or the surgical control console 16 may be in communication with the controller 20 via one or more I/O circuits 226. The I/O circuits may support various communication protocols including but not limited to Ethernet/IP, TCP/IP, Universal Serial Bus, Profibus, Profinet, Modbus, serial communications, etc.

In various implementations, the disclosure provides for a surgical camera system configured to capture image data indicative of a surgical implement comprising a fluorescent agent. The surgical camera system comprises a camera comprising at least one sensor configured to capture image data comprising a first range of wavelengths and a second range of wavelengths. An excitation light source emits an excitation emission at an excitation wavelength. A controller is in communication with the at least one sensor of the camera. The controller is configured process the image data from the at least one sensor and detect at least one fluorescent portion of the image data in response to a fluorescent emission generated by the fluorescent agent in the second range of wavelengths. The controller is further configured to generate enhanced image data demonstrating the at least one fluorescent portion of the surgical implement in the image data.

In various implementations, the systems and methods described in the application may comprise one or more of the following features or steps alone or in combination:
the first range of wavelengths comprises wavelengths from 400 nm to 650 nm in the visible light range;
the second range of wavelengths comprises wavelengths ranging from 650 nm to 900 nm in a near-infrared range;
the fluorescent emission is transmitted from the fluorescent agent at an output wavelength different from the excitation wavelength;
a visible light source that emits light in the first range of wavelengths;
the excitation light source, the visible light source, and the camera are incorporated in an endoscope;
the endoscope has a diameter of less than about 2 mm;
the at least one sensor of the camera comprises a plurality of sensors comprising a first sensor configured to capture first data in the first range of wavelengths and a second sensor configured to capture second data in the second range of wavelengths;
generate the enhanced image data by selectively applying an overlay defined by the second data from the second sensor over the first data from the first sensor;
the controller is further configured to determine a plurality of intensity levels of the fluorescent emission output from the at least one fluorescent portion generated by the fluorescent agent in the second range of wavelengths;
the controller is further configured to assign a distinctive color or pattern to each of the plurality of intensity levels; and/or
the enhancement of the image data comprises overlaying the distinctive color or pattern over the fluorescent portion demonstrating each of the plurality of intensity levels in the enhanced image data as the distinctive color or pattern.

In various implementations, the disclosure provides for method for displaying a surgical implement may comprise illuminating a fluorescent portion of the surgical implement in light comprising a first range of wavelengths corresponding to visible light and a second range of wavelengths comprising an excitation emission. The method may further include capturing first image data comprising the first range of wavelengths and capturing second image data comprising the second range of wavelengths demonstrating a fluorescent emission output from the fluorescent portion in response to the excitation emission. The method further includes generating enhanced image data demonstrating the first image data with at least one overlay or graphic demonstrating the fluorescent portion defined by the second image data overlaid on the first image data and communicating the enhanced image data for display on a display device.

In various implementations, the systems and methods described in the application may comprise one or more of the following features or steps alone or in combination:
placing the surgical implement in a surgical field;
targeting the surgical implement with the excitation emission;
detecting the fluorescent emission in the image data;
outputting an indication of the surgical implement detected in the image data in response to detecting the fluorescent emission;
displaying the detected fluorescent emission on a display as the overlay in a predefined pseudo-color;
the fluorescent emission emitted from the fluorescent portion is output at a wavelength different from the excitation wavelength;
identifying an intensity of the fluorescent emission output from the fluorescent portion generated by the fluorescent agent at a plurality of intensity levels;
assigning a distinctive color or pattern to each of the plurality of intensity levels;
the enhancement of the image data comprises overlaying the distinctive color or pattern over the fluorescent portion demonstrating each of the plurality of intensity levels in the enhanced image data;
detecting the fluorescent emission output from the fluorescent agent through a biological tissue; and/or
the excitation emission is transmitted through the biological tissue.

In some implementations, the disclosure provides for a surgical camera system configured to capture image data indicative of a surgical implement comprising a fluorescent agent. The surgical camera system comprises camera comprising at least one sensor configured to capture image data comprising a first range of wavelengths and a second range of wavelengths. An excitation light source emits an excitation emission at an excitation wavelength. A controller is in communication with the sensor of the camera. The controller is configured to process image data from the at least one image sensor comprising the first range of wavelengths and the second range of wavelengths and identify a plurality of intensity levels of at least one fluorescent emission output from the at least one fluorescent portion generated by the fluorescent agent in the second range of wavelengths. The controller is further configured to assign a distinctive color or pattern to each of the plurality of intensity levels and generate enhanced image data demonstrating the plurality of intensity levels of the fluorescent emission with the a distinctive colors or patterns. In some implementations, the enhancement of the image data comprises overlaying the distinctive color or pattern over the fluorescent portion demonstrating each of the plurality of intensity levels in the enhanced image data.

In some implementations, a surgical implement may comprise a body forming an exterior surface comprising a proximal end portion and a distal end portion. A fluorescent portion may comprise a fluorescent agent disposed on the exterior surface. The fluorescent portion may comprises at least one marking extending over the exterior surface and the fluorescent portion is configured to emit a fluorescent emission in a near-infrared range in response to an excitation emission.

In various implementations, the systems and methods described in the application may comprise one or more of the following features or steps alone or in combination:
the at least one marking of the fluorescent portion indicates at least one of a group consisting of: an identity of the surgical implement, an orientation of the surgical implement, and a dimension of the surgical implement;
the at least one marking comprises a plurality of graduated segments demonstrating a scale associated with a position or orientation of the surgical implement;
the at least one marking comprises a plurality of lateral graduated markings extending between the proximal end portion and the distal end portion;
the at least one marking comprises at least one longitudinal marking along a longitudinal axis between the proximal end portion and the distal end portion;
the at least one marking comprises one or more indicator symbols formed on the exterior surface by the fluorescent portion, wherein the indicator symbols comprise at least one of a pattern, shape, and alphanumeric character;
the indicator symbols identify a measurement unit or scale of the at least one marking;
the at least one marking is disposed within a groove or indentation formed in the exterior surface;
an orientation aperture of the fluorescent portion is exposed in the groove or indentation in response to an orientation of the surgical implement;
the orientation aperture is illuminated by the excitation emission based on the orientation of the surgical implement relative to a light source from which the excitation emission is output;
the orientation is identifiable based on an extent of the fluorescent emission projected through the aperture;
the light source is incorporated in an endoscope;
the fluorescent agent is an indocyanine green dye comprising an excitation wavelength of between about 600 nm and about 900 nm and an emission wavelength of about 830 nm;
the surgical implement is selected from the group consisting of: a suture, a pin, a screw, a plate, a surgical tool, and an implant; and/or
the surgical implement is selected from the group consisting of: a biter, grasper, retriever, pick, punch, hook, probe, elevator, retractor or scissors.

In some implementations, the surgical detection system may be configured to identify at least one surgical implement in an operating region. The system may comprise

a camera comprising at least one sensor configured to capture image data comprising a first range of wavelengths and a second range of wavelengths. An excitation light source emits an excitation emission at an excitation wavelength. A controller is in communication with the at least one sensor of the camera, the controller configured to process image data from the at least one sensor and identify the fluorescent emission in the image data output from at least one fluorescent portion of a surgical implement. The controller is further configured to detect a presence of the surgical implement in response to the presence of the fluorescent emission.

In various implementations, the systems and methods described in the application may comprise one or more of the following features or steps alone or in combination:
the fluorescent emission comprises a wavelength of light in the near-infrared range from approximately 650 nm to 900 nm;
the controller is further configured to detect a plurality of pixels in the image data in the near-infrared range corresponding to a location of the surgical implement;
the controller is further configured to identify the surgical instrument in response to at least one of a pattern, shape, and alphanumeric character of the plurality of pixels;
the controller is further configured to output an indication identifying the presence of the surgical implement;
the indication is output as a notification on a display device demonstrating the location of the surgical implement in the image data;
the controller is further configured to access a database comprising at least one computer vision template characterizing an appearance a potential surgical implement associated with a surgical procedure; and identify the potential surgical implement as the at least one surgical implement in response to the plurality of pixels in the near-infrared range corresponding to the computer vision template;
the controller is further configured to output a notification to a display device identifying a type or category of the at least one surgical implement in response to the identification associated with the computer vision template;
the surgical implement is selected from the group consisting of: a sponge, a suture, a pin, a screw, a plate, a surgical tool, and an implant;
the surgical implement is selected from the group consisting of: a biter, grasper, retriever, pick, punch, hook, probe, elevator, retractor, needle, or scissors;
the at least one surgical implement comprises a plurality of surgical implements and the at least one fluorescent emission comprises a plurality of fluorescent emissions output from the plurality of surgical implements, and wherein the controller is further configured to distinguish among the plurality of surgical implements in response to at least one of an intensity or a pattern of the fluorescent emissions output from the plurality of surgical implements; and/or
the plurality of surgical implements includes a plurality of sutures, and the controller is configured to distinguish between or among the plurality of sutures in response to characteristic patterns of fluorescent portions of the surgical implements.

In some implementations, the surgical camera system may be configured to capture image data indicative of a surgical implement comprising a fluorescent agent. The surgical camera system may comprise an endoscopic camera comprising at least one sensor configured to capture image data in a field of view comprising a first range of wavelengths and a second range of wavelengths. An excitation light source emits an excitation emission at an excitation wavelength. A controller is in communication with the sensor of the camera. The controller is configured to process the image data from the at least one sensor in the field of view depicting a cavity and detect a fluorescent emission output from at least one fluorescent portion of a surgical implement in the image data. The fluorescent emission is transmitted through a biological tissue forming at least a portion of the cavity. In response to a fluorescent emission, the controller generates enhanced image data demonstrating the at least one fluorescent portion of the surgical implement overlaid on the biological tissue depicted in the image data.

In various implementations, the systems and methods described in the application may comprise one or more of the following features or steps alone or in combination:
the excitation light source comprises an elongated shaft that forms a needle-shaped protrusion configured to output the excitation emission into the cavity;
the excitation light source is configured to output the excitation emission from a distal penetrating end of a needle that forms the elongated shaft;
the excitation light source originates from a first origin separate from a second origin of the field of view;
the excitation light source is separate from the endoscopic camera and each of the excitation light source and the endoscopic camera independently access the cavity;
the controller is further configured to detect the fluorescent emission transmitted through the biological tissue into the cavity in the image data;
the controller is further configured to output an indication identifying the presence of the fluorescent emission output from at least one fluorescent portion of a surgical implement in the image data;
the indication is output as the enhanced image data comprising an overlay over the image data demonstrating a location in the image data of the surgical implement embedded in the biological tissue;
the controller is further configured to output the enhanced image data to a display screen demonstrating the location of the surgical implement superimposed over the biological tissues as the overlay depicted in the image data;
the excitation light source emits light at an intensity ranging from about 0.1 mW/cm² to 1 W/cm², 0.5 mW/cm² to 500 mW/cm², 0.01 mW/cm² to 200 mW/cm², etc., and may vary significantly depending on the application and the emitter technology implemented;
the excitation emission is emitted at an excitation wavelength of between about 600 nm and about 900 nm and the fluorescent emission is output at an emission wavelength of about 830 nm.

There is disclosed in the above description and the drawings, a surgical camera system and method that fully and effectively overcomes the disadvantages associated with the prior art. However, it will be apparent that variations and modifications of the disclosed implementations may be made without departing from the principles described herein. The presentation of the implementations herein is offered by way of example only and not limitation, with a true scope and spirit being indicated by the following claims.

As used herein, words of approximation such as, without limitation, “approximately, “substantially,” or “about” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as having the required characteristics or capabilities of the unmodified feature. In general, but subject to the preceding discussion, a numerical value herein that is modified by a word of approximation such as “approximately” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

The claims:

1. A surgical camera system configured to capture image data indicative of a surgical implement comprising a fluorescent agent, the surgical camera system comprising:
a camera comprising at least one sensor configured to capture image data comprising a first range of wavelengths and a second range of wavelengths;
an excitation light source that emits an excitation emission at an excitation wavelength; and
a controller in communication with the at least one sensor of the camera, the controller configured to:
process the image data from the at least one sensor;
detect at least one fluorescent portion of the image data in response to a fluorescent emission generated by the fluorescent agent in the second range of wavelengths; and
generate enhanced image data demonstrating the at least one fluorescent portion of the surgical implement in the image data.

2. The surgical camera system according to claim 1, wherein the first range of wavelengths comprises wavelengths from 400 nm to 650 nm in the visible light range.

3. The surgical camera system according to claim 2, wherein the second range of wavelengths comprises wavelengths ranging from 650 nm to 900 nm in a near-infrared range.

4. The surgical camera system according to claim 1, wherein the fluorescent emission is transmitted from the fluorescent agent at an output wavelength different from the excitation wavelength.

5. The surgical camera system according to claim 1, further comprising:
a visible light source that emits light in the first range of wavelengths and wherein the excitation light source, the visible light source, and the camera are incorporated in an endoscope.

6. The surgical camera system according to claim 1, wherein the at least one sensor of the camera comprises a plurality of sensors comprising a first sensor configured to capture first data in the first range of wavelengths and a second sensor configured to capture second data in the second range of wavelengths.

7. The surgical camera system according to claim 6, wherein the controller is further configured to:
generate the enhanced image data by selectively applying an overlay defined by the second data from the second sensor over the first data from the first sensor.

8. The surgical camera system according to claim 1, wherein the controller is further configured to:
determine a plurality of intensity levels of the fluorescent emission output from the at least one fluorescent portion generated by the fluorescent agent in the second range of wavelengths.

9. The surgical camera system according to claim 8, wherein the controller is further configured to:
assign a distinctive color or pattern to each of the plurality of intensity levels.

10. The surgical camera system according to claim 9, wherein the enhancement of the image data comprises overlaying the distinctive color or pattern over the fluorescent portion demonstrating each of the plurality of intensity levels in the enhanced image data as the distinctive color or pattern.

11. A method for displaying a surgical implement, the method comprising:
illuminating a fluorescent portion of the surgical implement in light comprising a first range of wavelengths corresponding to visible light and a second range of wavelengths comprising an excitation emission;
capturing first image data comprising the first range of wavelengths;
capturing second image data comprising the second range of wavelengths demonstrating a fluorescent emission output from the fluorescent portion in response to the excitation emission;
generating enhanced image data demonstrating the first image data with at least one overlay or graphic demonstrating the fluorescent portion defined by the second image data overlaid on the first image data; and
communicating the enhanced image data for display on a display device.

12. The method according to claim 11, further comprising:
placing the surgical implement in a surgical field;
targeting the surgical implement with the excitation emission;
detecting the fluorescent emission in the image data; and
outputting an indication of the surgical implement detected in the image data in response to detecting the fluorescent emission.

13. The method according to claim 12, further comprising:
displaying the detected fluorescent emission on a display as the overlay in a predefined pseudo-color.

14. The method according to claim 11, further comprising:
identifying an intensity of the fluorescent emission output from the fluorescent portion generated by the fluorescent agent at a plurality of intensity levels.

15. The method according to claim 14, further comprising:
assigning a distinctive color or pattern to each of the plurality of intensity levels.

16. The method according to claim 15, wherein the enhancement of the image data comprises overlaying the distinctive color or pattern over the fluorescent portion demonstrating each of the plurality of intensity levels in the enhanced image data.

17. The method according to claim 11, further comprising:
detecting the fluorescent emission output from the fluorescent agent through a biological tissue.

18. The method according to claim 17, wherein the excitation emission is transmitted through the biological tissue.

19. A surgical camera system configured to capture image data indicative of a surgical implement comprising a fluorescent agent, the surgical camera system comprising:
a camera comprising at least one sensor configured to capture image data comprising a first range of wavelengths and a second range of wavelengths;
an excitation light source that emits an excitation emission at an excitation wavelength; and
a controller in communication with the sensor of the camera, the controller configured to:
process image data from the at least one image sensor comprising the first range of wavelengths and the second range of wavelengths;
identify a plurality of intensity levels of at least one fluorescent emission output from the at least one fluorescent portion generated by the fluorescent agent in the second range of wavelengths;
assign a distinctive color or pattern to each of the plurality of intensity levels; and
generate enhanced image data demonstrating the plurality of intensity levels of the fluorescent emission with the a distinctive colors or patterns.

20. The surgical camera system according to claim 19, wherein the enhancement of the image data comprises overlaying the distinctive color or pattern over the fluorescent portion demonstrating each of the plurality of intensity levels in the enhanced image data.
